(19) United States
(12) Patent Application Publication
(10) Pub. No.: US 2017/0251646 A1
(43) Pub. Date: Sep. 7, 2017

(54) TRANSGENIC PIGS LACKING ONE OR MORE CELLULAR TRANSPORT GENES

(71) Applicant: Indiana University Research and Technology Corporation, Indianapolis, IN (US)

(72) Inventor: A. Joseph Tector, Carmel, IN (US)

(21) Appl. No.: 15/446,742

(22) Filed: Mar. 1, 2017

Related U.S. Application Data
(60) Provisional application No. 62/301,706, filed on Mar. 1, 2016.

(51) Int. Cl. A01K 67/027

(52) U.S. Cl. CPC A01K 67/0276; A01K 2227/108; A01K 2267/025; A01K 2217/056

(57) ABSTRACT

The application provides methods of improving a medication related side effect in a human after transplant of transgenic organs, tissues or cells from transgenic pigs with a disrupted cellular transport gene or genes, and porcine organs, tissues, and cells therefrom are provided.

### CROSS-REFERENCES TO RELATED APPLICATIONS

This application claims priority to U.S. Provisional Application No. 62/301,706 filed on Mar. 1, 2016 entitled “Transgenic Pigs Lacking One or More Cellular Transport Genes,” the content of which is incorporated by reference in its entirety.

### STATEMENT REGARDING FEDERALLY SPONSORED RESEARCH OR DEVELOPMENT

N/A.

### FIELD OF THE INVENTION

The present invention relates generally to the field of xenotransplantation and genetic modification to develop transgenic pigs, transgenic porcine organs, tissue or cells suitable for transplant into a human, particularly to transgenic pigs lacking one or more cellular transport genes which thus exhibit decreased side effects from immunosuppressive medicines.

### BACKGROUND

It is well known that transplants from one animal into another animal of the same species, such as human to human, are a routine treatment option for many serious conditions including kidney, heart, lung, liver and other organ disease and skin damage such as severe burn disease. However, it is well known that there are not enough suitable organs available for transplant to meet current or expected clinical demands for organ transplants. Approximately 100,000 patients are on the kidney transplant list, and they remain on the waiting list an average of nearly five years before receiving a transplant or dying. In patients with kidney failure, dialysis increases the length of time the patient can wait for a transplant. More than 18,000 patients are on the UNOS liver transplant national waiting list, yet less than 7,000 transplants are performed annually in the United States. There is no system comparable to dialysis available for patients with liver disease or liver failure.

Xenotransplantation, the transplant of organs, tissues or cells from one animal into another animal of a different species, such as the transplantation of a pig organ into a human recipient has the potential to reduce the shortage of organs available for transplant, potentially helping thousands of people worldwide. However, xenotransplantation using standard, unmodified pig tissue into a human or other primate is accompanied by rejection of the transplanted tissue. The rejection may be a cellular rejection (lymphocyte mediated) or humoral (antibody mediated) rejection including but not limited to hyperacute rejection, an acute rejection, a chronic rejection, may involve survival limiting thrombocytopenia coagulopathy and an acute humoral xenograft reaction (AHXR). While not being limited by mechanism, both humoral and cellular rejection processes may target MHC molecules. The human hyperacute rejection response to pig antibodies present on transplanted tissue is so strong that the transplant tissue is typically damaged by the human immune system within minutes or hours of transplant into the human. Furthermore, different rejection mechanisms may predominate in an organ-preferred manner. An acute or rapid humoral rejection may begin within minutes of transplant; an acute or rapid cellular rejection may begin within days of the transplant. Both humoral and cellular rejections may also have a slower or chronic rejection phase; the chronic phases may occur for years. See Demetris et al. 1998 “Antibody-mediated Rejection of Human Orthotopic Liver Allografts. A study of liver transplantation across ABO blood group barriers”, Am J. Pathol 132:489-502; Nakamura et al 1993 “Liver allograft rejection in sensitized recipients. Observations in a Clinically Relevant Small Animal Model” Am J. Pathol. 142:1383-91; Furuya et al 1992. “Preformed Lymphocytotoxic Antibodies: the Effects of Class, Titer and Specificity on Liver v Heart Allografts” Hepatologyl6:1415-22; Tector et al 2001. “Rejection of Pig Liver Xenografts in Patients with Liver Failure: Implications for Xenotransplantation”, Liver Transpl pp.82-9; herein incorporated by reference in their entirety. For example, early development of thrombocytopenic coagulopathy is a major factor in non-human primate recipient death following xeno-transplant of a pig liver. Yet, if antibody mediated xenograft rejection is prevented, non-human primate (NHP) recipients of pig kidneys do not develop significant thrombocytopenia nor exhibit clinical manifestations of coagulopathy. See for example Ekser et al. 2012 “Genetically Engineered Pig to Baboon Liver Xenotransplantation: Histopathology of Xenografts and Native Organs” PLoS ONE pp e29720; Knosalla et al 2009, “Renal and Cardia Endothelial Heterogeneity Impact Acute Vascular Rejection in Pig to Baboon Xenotransplantation”, Am J Transplant 1006-16; Shimizu et al 2012. “Pathologic Characteristics of Transplanted Kidney Xenografts”, J. Am. Soc. Nephrology 225-35; herein incorporated by reference in their entirety.

Pig cells express α(1,3) galactosyltransferase (αGal) and cytidine monophosphate-N-acetylneuraminic acid hydroxylase (CMAH), which are not found in human cells. The αGal enzyme catalyzes the formation of galactose-α1,3-galactose (αGal) residues on glycoproteins. CMAH converts the sialic acid N-acetylneuraminic acid (Neu5Ac) to N-glycolylneuraminic acid (Neu5Gc). Antibodies to the Neu5Gc and αGal epitopes are present in human blood prior to implantation of the tissue, and are involved in the intense and immediate antibody mediated rejection of implanted tissue. Additionally pig cells express multiple swine leukocyte antigens (SLAs). Unlike humans, pigs constitutively express class I and class II SLA's on endothelial cells. SLAs and human leukocyte antigens (HLAs) share considerable sequence homology (Varela et al 2003 J. Am.Soc Nephrol 14:2677-2683). Porcine class 1 SLAs include antigens encoded by the SLA-1, SLA-2, SLA-3, SLA-4, SLA-5, SLA-9 and SLA-11 loci. Porcine class II SLA's include antigens encoded by the SLA-DQ and SLA-DR loci. Anti-HLA antibodies are present in human blood prior to implantation of porcine tissue and cross react with SLA antigens on porcine tissues.

The antibodies are present in the patient's blood prior to implantation of the tissue, contributing to the intense and immediate rejection of the implanted tissue. SLA antigens may also be involved with the T-cell mediated immune response.

Many strategies have been employed to address the rejection response including removing the genes encoding α(1,3) galactosyltransferase and CMAH to prevent expression of the enzymes, modifying the genes encoding α(1,3) galactosyltransferase and CMAH to reduce or limit expression of the enzymes, or otherwise limit the rejection response. U.S. Pat. No. 7,795,493 to Phelps et al describes a method for the production of a pig that lacks any expression of functional αGal. For instance, U.S. Pat. No. 7,547,816 to Day et al, describes a knockout pig with decreased expression of α(1,3) galactosyltransferase as compared to wild-type pigs. Although the Day pigs may have decreased expression of α(1,3) galactosyltransferase, Neu5Gc antigenic epitopes remain present and glycolipids from the Day pigs have αGal antigenic epitopes. Unfortunately, while the GTKO pig may have reduced anti-α-Gal antibodies as a barrier to xenotransplantation, studies using GTKO cardiac and renal xenografts in baboons show that the GTKO organs still trigger an immunogenic response, resulting in rejection or damage to the transplanted organ. Baboons transplanted with GTKO kidneys and treated with two different immunosuppressive regimens died within 16 days of surgery. Chen et al concluded “genetic depletion of Gal antigens does not provide a major benefit in xenograft survival” (Chen et al., (2005) Nature Med 11(12):1295-1298. U.S. Pat. No. 7,560,538 to Koike et al and U.S. Pat. Nos. 7,166,378 and 8,034,330 to Zhu et al describe methods for making porcine organs for transplantation that are less likely to be subject to delayed xenograft rejection and hyperacute rejection, respectively. Basnet et al examined the cytotoxic response of human serum to CMAH-/-mouse cells. Basnet et al concluded “the anti-Neu5Gc Ab-mediated immune response may be significantly involved in graft loss in xenogeneic cell transplantation, but not in organ transplantation” (Basnet et al., 2010 Xenotransplantation 17(6):440-448). Attempts to reduce the rejection response by adding multiple human proteins (human CD39, CD55, CD59 and fucosyltransferase) to Gal-knockout pigs had limited effect on extending kidney xenograft survival (LeBas-Bernardet et al 2011 Transplantation Proceedings 43:3426-30). Clearly progress in this field is critically dependent upon the development of genetically modified pigs.

Unfortunately, developing homozygous transgenic pigs is a slow process, requiring as long as three years using traditional methods of homologous recombination in fetal fibroblasts followed by somatic cell nuclear transfer (SCNT), and then breeding of heterozygous transgenic animals to yield a homozygous transgenic pig. The development of new transgenic pigs for xenotransplantation has been hampered by the lack of pluripotent stem cells, relying instead on the fetal fibroblast as the cell upon which genetic engineering was carried out. For instance, the production of the first live pigs lacking any functional expression of α(1,3) galactosyltransferase (GTKO) was first reported in 2003.

Additionally, a variety of immunosuppressive drugs are used to prevent organ rejection in organ transplant recipients. These immunosuppressive drugs including, but not limited to, tacrolimus (FK506) and cyclosporine, frequently induce deleterious side effects. The side effects of immunosuppressive drugs may include, but are not limited to, hypertension, renal tube dysfunction, hyperkalemia, hypercalciuria and acidosis in the transplant recipient. Some immunosuppressive drugs result in contraindications for other medications. Thus there is a need for transplant organs that are resistant to the side effects of the immunosuppressive drugs.

### BRIEF SUMMARY

This disclosure relates generally to methods of making porcine organs, tissues or cells with altered cellular transport gene expression for transplantation into a human. Cellular transport genes may include, but are not limited to the sodium chloride co-transporter (NCC) gene, FK12 (FK506) binding protein gene, NHE3, NHE1, megalin and cubulin.

A transgenic pig comprising a disrupted cellular transport gene in the nuclear genome of at least one cell is provided. Expression of a cellular transport gene in the transgenic pig is decreased as compared to expression in a wild-type pig. A porcine organ, tissue or cell obtained from the transgenic pig is provided. A porcine organ, tissue or cell may be selected from the group consisting of skin, heart, liver, kidneys, lung, pancreas, thyroid, small bowel and components thereof. In various embodiments, the cellular transport gene is selected from the group comprising NCC, NHE1, NHE3, megalin, cubulin, and FK506 binding protein 12. In an aspect, when tissue from the transgenic pig is transplanted into a human who is a recipient of an immunosuppressive medication, a side effect of the immunosuppressive medication is improved as compared to when tissue from a wild-type pig is transplanted into a human.

In various embodiments the altered side effect is selected from the group comprising high blood pressure, cardiomyopathy, renal tube dysfunction, hyperkalemia, hypercalciuria and acidosis. In an aspect of the application, when a heart from the transgenic pig is transplanted into a human recipient of FK506, the porcine heart exhibits less FK506-induced cardiomyopathy than a heart from a wildtype pig. In various embodiments when a kidney from the transgenic pig is transplanted into a human recipient of an immunosuppressive medication, the patient exhibits decreased kidney related side effects. In various aspects, the immunosuppressive medication is selected from the group comprising calcineurin inhibitors, FK506 and cyclosporine. In an aspect the application provides a transgenic pig wherein when a kidney from the transgenic pig is transplanted into a human and the human receives gentamycin, a gentamycin related nephrotoxicity symptom is improved as compared to when a kidney from a wild-type pig is transplanted into a human and the human receives gentamycin.

Aspects of the invention provide transgenic pigs with a disrupted cellular transport gene further comprising additional genetic modifications in the nuclear genome of at least one cell of the transgenic pig. Such additional genetic modifications may include disruption of the α(1,3)-galactosyltransferase gene, disruption of the CMAH gene, disruption of the ASGR1 gene and disruption of the β4GalNT2 gene, wherein the additional disruption or disruptions results in decreased expression of the gene of interest as compared to a wild-type pig.

### DETAILED DESCRIPTION OF THE INVENTION

The present application provides transgenic pigs and porcine organs, tissues and cells for transplantation into a human that do not express the indicated pig genome encoded products and methods of making and using the same. In one embodiment the application provides a transgenic pig comprising a disrupted cellular transport gene, wherein functional expression of said cellular transport gene in the transgenic pig is decreased as compared to a wild-type pig.

### I. In General

In the specification and in the claims, the terms “including” and “comprising” are open-ended terms and should be interpreted to mean “including, but not limited to . . . ” These terms encompass the more restrictive terms “consisting essentially of” and “consisting of”.

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. Compositions and Methods

Transgenic animals suitable for use in xenotransplantation and methods of producing mammals suitable for use in xenotransplantation are provided. Specifically, the present application describes the production of homozygous transgenic pigs with decreased expression of a cellular transport gene of interest.

In embodiments of the present invention, pigs and porcine organs, tissues and cells therefrom are provided in which a cellular transport gene is less active, such that the resultant cellular transport gene products no longer generate wild-type levels of a cellular transport polypeptide. In an alternative embodiment the cellular transport gene is inactivated in such a way that no transcription of the gene occurs. Methods of making transgenic pigs, and the challenges thereto, are discussed in Galli et al 2010 Xenotransplantation 17(6) p.397-410 and in Perkel 2016 Nature Biotechnology 34:1. Methods and cell cultures of the invention are further detailed below herein.

The term “transgenic mammal” refers to a mammal wherein a given gene has been altered, removed or disrupted. It is to be emphasized that the term is to be intended to include all progeny generations. Thus, the founder animal and all F1, F2, F3 and so on progeny thereof are included, regardless of whether progeny were generated by somatic cell nuclear transfer (SCNT) from the founder animal or a progeny animal or by traditional reproductive methods. By “single transgenic” is meant a transgenic mammal wherein one gene has been altered, removed or disrupted. By “double transgenic” is meant a transgenic mammal wherein two genes have been altered, removed or disrupted. By “triple transgenic” is meant a transgenic mammal wherein three genes have been altered, removed or disrupted. By “quadruple transgenic” is meant a transgenic mammal wherein four genes have been altered, removed or disrupted.

In principle transgenic animals may have one or both copies of the gene sequence of interest disrupted. In the case where only one copy or allele of the nucleic acid sequence of interest is disrupted, the transgenic animal is termed a “heterozygous transgenic animal”. The term “null” mutation encompasses both instances in which the two copies of a nucleotide sequence of interest are disrupted differently but for which the disruptions overlap such that some genetic material has been removed from both alleles, and instances in which both alleles of the nucleotide sequence of interest share the same disruption. In various embodiments disruptions of the three genes of interest may occur in at least one cell of the transgenic animal, at least a plurality of the animal's cells, at least half the animal's cells, at least a majority of animal's cells, at least a supermajority of the animal's cells, at least 70%, 75″, 80%, 85%, 90%, 95%, 98%, or 99% of the animal's cells.

The term “chimera”, “mosaic” or “chimeric mammal” refers to a transgenic mammal with a transgenic in some of its genome-containing cells. A chimera has at least one cell with an unaltered gene sequence, at least several cells with an unaltered gene sequence or a plurality of cells with an unaltered sequence.

The term “heterozygote” or “heterozygotic mammal” refers to a transgenic mammal with a disruption on one of a chromosome pair in all of its genome containing cells.

The term “homozygote” or “homozygotic mammal” refers to a transgenic mammal with a disruption on both members of a chromosome pair in all of its genome containing cells. A “homozygous alteration” refers to an alteration on both members of a chromosome pair.

A “non-human mammal” of the application includes mammals such as rodents, sheep, dogs, ovine such as sheep, bovine such as beef cattle and milk cows, and swine such as pigs and hogs. Although the application provides a typical non-human animal (pigs), other animals can similarly be genetically modified.

A “mutation” is a detectable change in the genetic material in the animal that is transmitted to the animal's progeny. A mutation is usually a change in one or more deoxyribonucleotides, such as, for example adding, inserting, deleting, inverting or substituting nucleotides.

By “pig” is intended any pig known to the art including, but not limited to, a wild pig, domestic pig, mini pigs, a Sus scrofa pig, a Sus scrofa domesticus pig, as well as in-bred pigs. Without limitation the pig can be selected from the group comprising Landrace, Yorkshire, Hampshire, Duroc, Chinese Meishan, Chester White, Berkshire Goettingen, Landrace/York/Chester White, Yucatan, Bama Xiang Zhu, Wuzhishan, Xi Shuang Banna and Pietrain pigs. Porcine organs, tissues or cells are organs, tissues, devitalized animal tissues, or cells from a pig.

The alpha 1,3 galactosyltransferase (αGal, GGTA, GGT1, GT, αGT, GGTA1, GGTA-1) gene encodes an enzyme (GT, αGal, α1,3 galactosyltransferase). Ensemble transcript ENSSSCG00000005518 includes the porcine GGTA1 nucleotide sequence. Functional α1,3 galactosyltransferase catalyzes formation of galactose-α1,3-galactose (αGal,Gal,Gal, gal1,3gal, gal1-3gal) residues on glycoproteins. The galactose-α1,3-galactose (αGal) residue is an antigenic epitope or antigen recognized by the human immunological system. Removing αGal from transgenic organ material does not eliminate the human immunological response to transplant of foreign material, suggesting an involvement of additional antibodies in the rapid immunological response to xenotransplant. (Mohiudden et al (2014), Am J. Transplantation 14:488-489 and Mohiudden et al 2014 Xenotransplantation 21:35-45). Disruptions of the αGal gene that result in decreased expression of functional αGal may include but are not limited to a 3 base pair deletion adjacent to a G to A substitution, a single base pair deletion, a single base pair insertion, a two base pair insertion, a six base pair deletion, a ten base pair deletion, a seven base pair deletion, an eight base pair insertions for a five base pair deletion and a five base pair insertion (see Table 1). The Crispr target sequence is in exon 3 of the gene, near the start codon. Relevant disruptions of the αGal gene are known in the art and may include but are not limited to those provided in U.S. patent application Ser. No: 14/436,963, PCT US15/56730, and 62/185996.

Swine produce swine leukocyte antigens (SLA) from multiple SLA genes. Humans and non-human primate CD8+ and CD4+ T cells can be activated by SLA Class I and II, respectively. SLA's are characterized in a class selected from the group comprising Class I and Class II. SLA genes include, but are not limited to SLA-1, SLA-2, SLA-3, SLA-4, SLA-5, SLA-9, SLA-11, SLA-DQ and SLA-DR. SLA-1, SLA-2 and SLA-3 are SLA Class I (SLA1) genes. SLA-DQ and SLA-DR are SLA Class II genes. Anti-SLA class 1 (anti-SLA1) antibodies may react with products of the SLA-1, SLA-2 and SLA-3 genes. The SLA-1*0702 allele sequence is available as Genbank Acc. No: EU440330.1. The SLA-1*1201 allele sequence is available as Genbank Acc. No: EU440335.1. The SLA-1*1301 allele sequence is available as Genbank Acc. No: EU440336.1. The SLA-2 1001 allele sequence is available as Genbank Acc. No: EU432084.1. The SLA-2 2002 allele sequence is available as Genbank Acc. No: EU432081.1. The SLA-3*0402 allele sequence is available as Genbank Acc. No: EU432092.1. The SLA-3*0502 allele sequence is available as Genbank Acc. No: EU432094.1. Transgenic pigs expressing a dominant negative version of the human class I transactivator (CIITA), a transcription factor critical for expression of SLA class II have been created. The CIITA expressing pigs appeared healthy and viable. In the CIITA pigs, class II SLA expression was reduced by 40-50%. See Hara et al 2013, “Human dominant-negative class II transactivator transgenic pigs-effect on the human anti-pig T-Cell immune response and immune status”, Immunol 140:39-46, herein incorporated by reference in their entirety. Relevant disruptions of the SLA-I gene are known in the art and may include but are not limited to those provided in U.S. patent application Ser. No: 14/436,963, PCT US15/56730, and 62/185996.

The cytidine monophosphate-N-acetylneuraminic acid hydroxylase (CMP-Neu5Ac hydroxylase gene, CMAH) gene encodes an enzyme (CMAH). Functional CMAH catalyzes conversion of sialic acid N-acetylneuraminic acid (Neu5Ac) to N-glycolylneuraminic acid (Neu5Gc). The Neu5Gc residue is an antigenic epitope or antigen recognized by the human immunological system. The Ensembl database id Gene: ENSSSCG00000001099 includes the porcine CMAH nucleotide sequence. The Crispr target area is near exon 6. Disruptions of the CMAH gene that result in decreased expression of functional CMAH may include but are not limited to a four base pair insertion, a one base pair deletion, a two base pair deletion, a three base pair deletion, a five base pair deletion, an eight base pair deletion, an eleven base pair deletion, a twelve base pair deletion, a single base pair insertion, a two base pair insertion for single base pair deletion, and a three base pair deletion for a five base pair insertion. Relevant disruptions of the CMAH gene are known in the art and may include but are not limited to those provided in U.S. patent application Ser. No: 14/436,963, PCT US15/56730, and 62/185996.

Transgenic Animals. The present invention provides a transgenic animal lacking any expression of a cellular transport gene of interest. The animal can be any mammal suitable for xenotransplantation. In a specific embodiment, the animal is a pig. A triple transgenic product or pig may be created in a CMAH/αGal double knockout background. A transgenic pig lacking a cellular transport gene of interest may be created in a wildtype or transgenic background.

The phrase “disrupted gene” is intended to encompass insertion, interruption, or deletion of a nucleotide sequence of interest wherein the disrupted gene either encodes a polypeptide having an altered amino acid sequence that differs from the amino acid sequence of the endogenous sequence, encodes a polypeptide having fewer amino acid residues than the endogenous amino acid sequence or does not encode a polypeptide although the nucleotide sequence of interest encodes a polypeptide.

The present specification provides a transgenic animal with reduced expression of a functional cellular transport genes. In one embodiment the transgenic animal lacks functional expression of a cellular transport gene of interest. In an embodiment the transgenic animal has reduced expression of additional genes of interest including but not limited to functional αGal, SLA, B4GalNT2 and CMAH genes and combinations thereof. Transgenic pigs may be further altered to express inhibitory or co-inhibitory molecules or by removing additional molecules including but not limited to ASGR1, vWF, Mac-1 (CR3, complement receptor 3), CD11b or CD18 or by providing increased expression of a Class I HLA gene.

The present invention provides a transgenic animal with decreased expression of a cellular transport gene.

Transgenic transplant material. Transplant material encompasses organs, tissue and/or cells from an animal for use as xenografts. Transplant material for use as xenografts may be isolated from transgenic animals with decreased expression of a cellular transport gene. Transgenic transplant material from transgenic pigs can be isolated from a prenatal, neonatal, immature or fully mature animal. The transplant material may be used as temporary or permanent organ replacement for a human subject in need of an organ transplant. Any porcine organ can be used including, but not limited to, the brain, heart, lung, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, small bowel, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes and lymph vessels.

In another embodiment, the application provides non-human tissues that are useful for xenotransplantation. In various embodiments, the non-human tissue is porcine tissue from a transgenic pig lacking a functional cellular transport gene of interest. Any porcine tissue can be used including but not limited to, epithelium, connective tissue, blood, bone, cartilage, muscle, nerve, adenoid, adipose, areolar, brown adipose, cancellous muscle, cartilaginous, cavernous, chondroid, chromaffin, dartoic, elastic, epithelial, fatty, fibrohyaline, fibrous, Gamgee, gelatinous, granulation, gut-associated lymphoid, skeletal muscle, Haller's vascular, indifferent, interstitial, investing, islet, lymphatic, lymphoid, mesenchymal, mesonephric, multilocular adipose, mucous connective, myeloid, nasion soft, nephrogenic, nodal, osteoid, osseus, osteogenic, retiform, periapical, reticular, smooth muscle, hard hemopoietic and subcutaneous tissue, devitalized animal tissues including heart valves, skin, and tendons, and vital porcine skin.

Another embodiment provides cells and cell lines from porcine transgenic animals with reduced or decreased expression of a cellular transport gene. In one embodiment these cells or cell lines can be used for xenotransplantation. Cells from any porcine tissue or organ can be used including, but not limited to: epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, granulosa cells, cumulus cells, epidermal cells, endothelial cells, Islet of Langerhans cells, pancreatic insulin secreting cells, pancreatic alpha-2 cells, pancreatic beta cells, pancreatic alpha-1 cells, bone cells, bone precursor cells, neuronal stem cells, primordial stem cells, hepatocytes, aortic endothelial cells, microvascular endothelial cells, umbilical vein endothelial cells, fibroblasts, liver stellate cells, aortic smooth muscle cells, cardiac myocytes, neurons, Kupffer cells, smooth muscle cells, Schwann cells, erythrocytes, platelets, neutrophils, lymphocytes, monocytes, eosinophils, basophils, adipocytes, chondrocytes, pancreatic islet cells, thyroid cells, parathyroid cells, parotid cells, glial cells, astrocytes, red blood cells, white blood cells, macrophages, somatic cells, pituitary cells, adrenal cells, hair cells, bladder cells, kidney cells, retinal cells, rod cells, cone cells, heart cells, liver cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, dopaminergic cells, squamous epithelial cells, osteocytes, osteoblasts, osteoclasts, embryonic stem cells, fibroblasts and fetal fibroblasts.

In an embodiment the application provides non-human material suitable for transfusions from transgenic porcine animals with reduced expression of a cellular transport gene. These materials suitable for transfusions may include, but are not limited to, blood, whole blood, plasma, serum, red blood cells, platelets, and white bloods cells. Such materials may be isolated, enriched or purified. Methods of isolating, enriching or purifying material suitable for transfusion are known in the art.

Nonviable derivatives include tissues stripped of viable cells by enzymatic or chemical treatment these tissue derivatives can be further processed through crosslinking or other chemical treatments prior to use in transplantation. In a preferred embodiment, the derivatives include extracellular matrix derived from a variety of tissues, including skin, bone, urinary, bladder or organ submucosal tissues. In addition, tendons, joints, and bones stripped of viable tissue to including but not limited to heart valves and other nonviable tissues as medical devices are provided. In an embodiment, serum or medium suitable for cell culture and isolated from a transgenic pig of the invention are provided. Components of porcine transgenic organs, tissues or cells are also provided. Components may also be modified through any means known in the art including but not limited to crosslinking and aldehyde crosslinking. Components may vary depending on the larger organ or tissue from which the component is obtained. Skin components may include but are not limited to stripped skin, collagen, epithelial cells, fibroblasts and dermis. Bone components may include but are not limited to collagen and extracellular matrix. Heart components may include but are not limited to valves and valve tissue.

“Xenotransplantation” encompasses any procedure that involves the transplantation, implantation or infusion of cells, tissues or organs into a recipient subject from a different species. Xenotransplantation in which the recipient is a human is particularly envisioned. Thus xenotransplantation includes but is not limited to vascularized xenotransplant, partially vascularized xenotransplant, unvascularized xenotransplant, xenodressings, xenobandages, xenotransfusions, and xenostructures.

In embodiments, cell culture reagents isolated from a transgenic pig comprising a disrupted cellular transport gene are provided. Cell culture reagents are reagents utilized for tissue culture, in vitro tissue culture, microfluidic tissue culture, cell culture or other means of growing isolated cells or cell lines. Cell culture reagents may include but are not limited to cell culture media, cell culture serum, a cell culture additive, a feeder cell, and an isolated cell capable of proliferation. By an “isolated cell capable of proliferation” is intended a cell isolated or partially isolated from other cell types or other cells wherein the cell is capable of proliferating, dividing or multiplying into at least one additional clonal cell.

Cells grown in culture may synthesize or metabolically incorporate antigenic epitopes into a compound of interest produced by the cultured cell. The antigenic epitopes may result in increased binding by human antibodies and decreased efficacy of the compound of interest. See Ghaderi et al 2010 Nature Biotechnology 28(8):863-867, herein incorporated by reference in its entirety. Growing the producing cell in a cell culture reagent with an altered epitope profile such as a reduced level of a cellular transport gene may reduce the level of cellular transport antigens combined on the compound of interest. Compounds of interest may include but are not limited to glycoproteins and glycolipids. Glycoproteins of interest may include but are not limited to an antibody, growth factor, cytokine, hormone or clotting factor. Glycolipids of interest may include but are not limited to therapeutics, antigens, and bio-surfactants.

The word “providing” is intended to encompass preparing, procuring, getting ready, making ready, supplying or furnishing. It is recognized that methods of providing a cell may differ from methods of providing a subject, methods of providing an organ may differ from methods of providing a pig, methods of providing a kidney may differ from methods of providing a liver and methods of providing an organ may differ from methods of providing a material suitable for transfusion.

Transplant rejection occurs when transplanted tissue, organs, cells or material are not accepted by the recipients body. In transplant rejection, the recipient's immune system attacks the transplanted material. Multiple types of transplant rejection exist and may occur separately or together. Rejection processes included but are not limited to hyperacute rejection (HAR), acute humoral xenograft rejection reaction (AHXR), thrombocytopenia, acute humoral rejection, hyperacute vascular rejection, antibody mediated rejection and graft versus host disease. By “hyperacute rejection” we mean rejection of the transplanted material or tissue occurring or beginning within the first 24 hours post-transplant involving one or more mechanisms of rejection. Rejection encompasses but is not limited to “hyperacute rejection”, “humoral rejection”, “acute humoral rejection”, “cellular rejection” and “antibody mediated rejection”. The acute humoral xenograft reaction (AHXR) is characterized by a spectrum of pathologies including, but not limited to, acute antibody mediated rejection occurring within days of transplant, the development of thrombotic microangiopathy (TMA), microvascular angiopathy, pre-formed non-Gal IgM and IgG binding, complement activation, microvascular thrombosis and consumptive thrombocytopenia within the first few weeks post transplant. Thrombocytopenia is a quantity of platelets below the normal range of 140,000 to 440,000/μl. Thrombocytopenia related symptoms include, but are not limited to, internal hemorrhage, intracranial bleeding, hematuria, hematemesis, bleeding gums, abdominal distension, melena, prolonged menstruation, epistaxis, ecchymosis, petechiae or purpura. Uptake of human platelets by pig livers contributes to the development of thrombocytopenia in xenograft recipients. Thrombocytopenia may occur upon reperfusion of the xenotransplanted organ or after the immediate post-reperfusion period.

Rejection related symptoms include but are not limited to hyperacute rejection related symptoms and acute humoral xenograft reaction related symptoms. Rejection related symptoms may include, but are not limited to, thrombotic microangiopathy (TMA), microvascular angiopathy, pre-formed non-Gal IgM and IgG binding, complement activation, agglutination, fibrosis, microvascular thrombosis, consumptive thrombocytopenia, consumptive coagulopathy, profound thrombocytopenia, refractory coagulopathy, graft interstitial hemorrhage, mottling, cyanosis, edema, thrombosis, necrosis, fibrin thrombi formation, systemic disseminated intravascular coagulation, IgM deposition in glomerular capillaries, IgG deposition in glomerular capillaries, elevated creatinine levels, elevated BUN levels, T cell infiltrate, infiltrating eosinophils, infiltrating plasma cells, infiltrating neutrophils, arteritis, antibody binding to endothelium, altered expression of ICOS, CTLA-4, BTLA, PD-1, LAG-3, or TIM-3, and systemic inflammation.

“Hyperacute rejection related symptom” is intended to encompass any symptom known to the field as related to or caused by hyperacute rejection. It is recognized that hyperacute rejection related symptoms may vary depending upon the type of organ, tissue or cell that was transplanted. Hyperacute rejection related symptoms may include, but are not limited to, thrombotic occlusion, hemorrhage of the graft vasculature, neutrophil influx, ischemia, mottling, cyanosis, edema, organ failure, reduced organ function, necrosis, glomerular capillary thrombosis, lack of function, hemolysis, fever, clotting, decreased bile production, asthenia, hypotension, oliguria, coagulopathy, elevated serum aminotransferase levels, elevated alkaline phosphatase levels, jaundice, lethargy, acidosis and hyperbilirubenemia and thrombocytopenia.

Platelets, also known as thrombocytes, are enucleate fragments of megakaryocytes involved in blood coagulation, hemostasis and blood thrombus formation. Human platelets are routinely isolated through a variety of methods including, but not limited to, platelet apheresis, plateletpheresis and ultracentrifugation. The phrase “platelet uptake” is intended to encompass the incorporation of a platelet into a liver or liver cell. While not being limited by mechanism, such uptake may occur through a phagocytic process. Platelet uptake may be monitored by any platelet uptake monitoring assay known in the art. Platelet uptake monitoring assays include, but are not limited to immunological methods, western blots, immunoblotting, microscopy, confocal microscopy, transmission electron microscopy and phagosome isolation. It is recognized that the appropriate platelet uptake monitoring assay may depend upon the type of label used. Platelet uptake may be measured as a percentage of total platelets absorbed, percentage of total platelets not absorbed, a ratio of absorbed to unabsorbed platelets, percentage of cells absorbing at least one platelet, percentage of cells not absorbing a platelet, or number of platelets absorbed per cell. It is recognized that platelet uptake by more than one cell type may contribute to the total platelet uptake of the liver. Total platelet uptake by an animal liver may include platelet uptake by liver sinusoidal endothelial cells, platelet uptake by Kuppffer cells, platelet uptake by LSECs and Kupffer cells and platelet uptake by additional cell types. It is recognized that platelet uptake by different cell types may contribute similar or disparate fractions of the total platelet uptake by a liver. Thus an alteration, inhibition, reduction, decrease, or lowering of platelet uptake by a liver comprises an alteration, inhibition, reduction, decrease, or lowering of platelet uptake by one or more liver cell types. While not being limited by mechanism, platelet uptake may occur through phagocytosis by LSEC and Kupffer cells. Phagocytosis is characterized by the formation of an endosome which by the fusion of lysosomes containing degradative enzymes becomes a phagosome.

Any method of evaluating, assessing, analyzing, measuring, quantifying, or determining a rejection related symptom known in the art may be used with the claimed compositions and methods. Methods of analyzing a rejection related symptom may include, but are not limited to, laboratory assessments including CBC with platelet count, coagulation studies, liver function tests, flow cytometry, immunohistochemistry, standard diagnostic criteria, immunological methods, western blots, immunoblotting, microscopy, confocal microscopy, transmission electron microscopy, IgG binding assays, IgM binding assays, expression asays, creatinine assays and phagosome isolation.

Transplant rejection of human organs, tissues and cells is managed through close matching of donor and recipient types prior to transplant and immunosuppressive therapy prior to and after transplant. Xenotransplant presents additional challenges which have been thought to only add to the complexity of the induction and maintenance immunosuppressive protocols and acute rejection response protocols. Each immunosuppressive compound or agent may result in one or more deleterious side effects. Careful consideration and evaluation of the benefits of each immunosuppressive compound versus the deleterious side effects is required. A transgenic pig with an altered cellular transport gene such that the transgenic pig is resistant to one or more immunosuppressive compounds thus preventing a transplanted organ, tissue or cell from the transgenic pig from importing the immunosuppressive agent and decreasing the side effects caused by the immunosuppressive agent is provided. For example FK506 is a commonly used immunosuppressive agent. Heart transplant patients treated with a FK506 are at increased risk for FK506-related cardiomyopathy. Providing a donor heart resistant to FK506 may reduce the risk of FK506-related cardiomyopathy. A side effect of FK506 is increased blood pressure related to stimulation of the renal sodium chloride cotransporter gene (NCC). The NCC gene was knocked out of mice and FK506 was administered. The NCC knockout mice did not experience the increase in blood pressure normally associated with FK506 administration. See for example Hoorn et al, 2011 Nature Medicine vol. 17, no.11, 1304-1309, herein incorporated by reference in its entirety. Transplanting FK506 resistant kidneys into a patient in need of a kidney transplant reduces the increase in blood pressure associated with FK506 treatment. Additionally the renal side effects associated with calcineurin inhibitors result in a contraindication for gentamycin use. Gentamycin is a well-regarded antibiotic whose use may be desirable in a transplant patient. Transplanting a kidney resistant to a calcineurin inhibitor rather than a wild-type kidney would allow the transplant patient to be treated with gentamycin when desired. Side effects related to immunosuppressive agents may include, but are not limited to, hypertension, hyperkalemia, cardiomyopathy, hypercalciuria, acidosis, renal tube dysfunction and medication contraindications.

By “improving”, “bettering”, “ameliorating”, “enhancing”, and “helping” is intended advancing or making progress in what is desirable. It is also envisioned that improving a side effect may encompass a decrease, lessening, or diminishing of an undesirable side effect. It is further recognized that a side effect may be improved while another side effect is altered. The altered second side effect may be improved or increased. A second altered side effect may be altered in a less desirable manner.

Expression of a gene product is decreased when total expression of the gene product is decreased, a gene product of an altered size is produced or when the gene product exhibits an altered functionality. Thus if a gene expresses a wild-type amount of product but the product has an altered enzymatic activity, altered size, altered cellular localization pattern, altered receptor-ligand binding or other altered activity, expression of that gene product is considered decreased. Expression may be analyzed by any means known in the art including, but not limited to, RT-PCR, Western blots, Northern blots, microarray analysis, immunoprecipitation, radiological assays, polypeptide purification, spectrophotometric analysis, Coomassie staining of acrylamide gels, ELISAs, 2-D gel electrophoresis, in situ hybridization, chemiluminescence, silver staining, enzymatic assays, ponceau S staining, multiplex RT-PCR, immunohistochemical assays, radioimmunoassay, colorimetric assays, immunoradiometric assays, positron emission tomography, fluorometric assays, fluorescence activated cell sorter staining of permeablized cells, radioimunnosorbent assays, real-time PCR, hybridization assays, sandwich immunoassays, flow cytometry, SAGE, differential amplification or electronic analysis. Expression may be analyzed directly or indirectly. Indirect expression analysis may include but is not limited to, analyzing levels of a product catalyzed by an enzyme to evaluate expression of the enzyme. See for example, Ausubel et al, eds (2013) Current Protocols in Molecular Biology, Wiley-Interscience, New York, N.Y. and Coligan et al (2013) Current Protocols in Protein Science, Wiley-Interscience New York, NY.

“As compared to” is intended to encompass comparing something to a similar but separate thing, such as comparing a data point obtained from an experiment with a transgenic pig to a data point obtained from a similar experiment with a wildtype pig. The word “comparing” is intended to encompass examining character, qualities, values, quantities, or ratios in order to discover resemblances or differences between that which is being compared. Comparing may reveal a significant difference in that which is being compared. By “significant difference” is intended a statistically significant difference in results obtained for multiple groups such as the results for material from a transgenic pig and material from a wild-type pig or results for material from a triple transgenic product or pig and material from a double transgenic product or pig. Generally statistical significance is assessed by a statistical significance test such as but not limited to the student's t-test, Chi-square, one-tailed t-test, two-tailed t-test, ANOVA, Dunett's post hoc test, Fisher's test and z-test. A significant difference between two results may be results with a p<0.1, p<0.05, p<0.04, p<0.03, p<0.02, p<0.01 or greater.

The word “isolated” is intended to encompass an entity that is physically separated from another entity or group. An isolated cell is physically separated from another group of cells. Examples of a group of cells include, but are not limited to, a developing cell mass, a cell culture, a cell line, a tissue, an organ and an animal. The word “isolating” is intended to encompass physically separating an entity from another entity or group. Examples include physically separating a cell from other cells, physically separating a cell component from the remainder of the cell and physically separating tissue or organ from an animal. An isolated cell or cell component is separated by 10%, 15%, 20%, 25%, 30%, 35%, 40%, 45%, 50%, 55%, 60%, 65%, 70%, 75%, 80%, 85%, 90%, 95%, 96%, 97%, 98%, 99%, up to 100% of other naturally occurring cells or cell components. Methods for isolating one or more cells from another group of cells are known in the art. See for example Freshney (ED) Culture of Animal Cells: a manual of basic techniques (3^{rd} Ed.) 1994, Wiley-Liss; Spector et al (Eds)(1998) Cells: a Laboratory Manual (vol.1) Cold Spring Harbor Laboratory Press and Darling et al (1994) Animal Cells: culture and media John Wiley & Sons. Methods of isolating a tissue or an organ from an animal are known in the art and vary depending on the tissue or organ to be isolated and the desired method of transplanting the tissue or organ. Methods of isolating a transfusion product from an animal or sample are known in the art and vary depending on the desired transfusion product. Such methods include but are not limited to centrifugation, dialysis, elution, apheresis and cryoprecipitation.

By “surgically attaching” is intended joining, combining, uniting, attaching, fastening, connecting, joining or associating through any surgical method known in the art.

The efficiency of producing genetically modified pigs increases when SCNT is performed primarily with genetically modified cells. The process of making genetic modifications in pig cells is less than 100% efficient. Phenotypic sorting of targeted cells simplifies the process of isolating modified cells from the whole population of cells. Methods of phenotypic sorting include, but are not limited to, confocal microscopy, flow cytometry, Western blotting, RT-PCR, 1B4 lectin binding and co-enrichment. It is understood that not all methods of phenotypic sorting are suitable for all genetic target modfications. Counter-selection with IB4 lectin binding is particularly useful for modifications of the αGal gene. Further counter-selection with IB4 lectin binding is particularly useful for multiple modifications, when at least one target is the αGal gene.

In yet another aspect, the present invention provides a method for producing viable pigs lacking any functional expression of a cellular transport gene. In one embodiment, the pigs are produced as described below. Methods of making transgenic pigs, and the challenges thereto, are discussed in Galli et al. 2010 Xenotransplantation, 17(6) p. 397-410, incorporated by reference herein for all purposes.

The following Examples are offered for illustrative purposes only and are not intended to limit the scope of the present invention in any way. Indeed various modifications in addition to those shown and described herein will become apparent to those skilled in the art from the foregoing description and the following examples and fall within the scope of the appended claims.

### EXAMPLES

### Example 1

### Design of Targeting Vectors

A Crispr construct with a sequence that is the reverse complement of a portion of the target sequence is created and utilized in the creation of a transgenic product.

The designed annealed oligonucleotides are cloned into a plasmid such as pX330 to generate gRNA using the CRISPR-associated Cas9 nuclease system. One microgram pX330 is digested with Bbsl (New England Biolabs, Ipswich Mass.) for 30 minutes at 37° C. Each pair of phosphorylated oligonucleotides is annealed using a Veriti thermocycler (Applied Biosystems, Grand Island N.Y.) starting at 37° C. for 30 minutes, followed by a step at 95° C. for 5 min and then ramp down to 25° C. at 5° C./min. Digested pX330 is ligated to the annealed pair of oligonucleotides for 10 minutes at room temperature. Ligation reaction is used to transform TOP10 competent cells (Invitrogen), following the manufacturer's protocol. The QIAPrep kit (Qiagen Valencia Calif.) is used to isolated plasmid from 15 colonies per treatment. DNA clones are sequenced and used to transfect porcine fetal fibroblasts.

### Example 2

### Production of Transgenic Cells

Fetal fibroblast cells from a pig are used in this study (See for example Reyes et al (2014), Tissue Antigens 84(5):484-488, herein incorporated by reference in its entirety). Fetal fibroblasts cultured in stem cell media (FFSCs) are resuspended and cultured in MEM-α (Invitrogen, Carlsbad, Calif.)/EGM-MV (Lonza, Basel, Switzerland) media supplemented with 10% FBS (HyClone, Logan Utah), 10% horse serum (Invitrogen), 12 mM HEPES (Sigma-Aldrich, St. Louis Mo.), and 1% penicillin/streptomycin (Life Technologies, Grand Island N.Y.) and are cultured on collagen-I-coated plates (Becton Dickinson, Bedford Mass.) at 38.5° C., 5% CO₂ and 10% O₂. The cells are treated with target-specific gRNA and Cas9.

FFSC's are seeded in early passage (passage 2) onto six-well plates 24 hours before transfection. Cells are harvested and counted and 1×10⁶ cells are resuspended in 800 μl fresh sterile electroporation buffer (75% cytosalt buffer: 120 mM KCl, 0.15 mM CaCl₂, 10 mM K₂HPO₄ [pH 7.6], 5 mM MgCl₂) and 25% Opti-Mem (Life Technologies). Cells are mixed with 2 μg plasmid DNA in 4 mm cuvettes. Transfection is performed using the Gene Pulser Xcell (Bio-Rad, Hercules, Calif.) following the manufacturer's recommended protocols for mammalian cells. Treated cells are seeded onto six-well plates and grown until confluent. Cell screening is performed using a BD Accuri C6 flow cytometer (BD Biosciences, San Jose Calif.) using target specific antibodies. Cells with low expression for the cellular transport gene of interest are expanded and analyzed by FACS.

### Example 3

### Flow Cytometry Analysis

For flow cytometry, porcine PBMCs are prepared using Ficoll-Paque Plus as described elsewhere (See Lutz et al, 2013 Xenotransplantation 20:27-35, herein incorporated by reference in its entirety). PBMC are stained with appropriate antibodies. Dead cells are excluded from analysis using fixable viability dye eFluor 660 (eBioscience, San Diego Calif.). Analysis is performed using an Accuri C6 flow cytometer and CFlow software (Accuri, Ann Arbor Mich.) and FlowJo software (TreeStar, Ashland Oreg.).

Primary kidney endothelial cells are isolated using 0.025% collagenase type IV from Clostridium histolyticum (Sigma-Aldrich) and cultured for 3 days in RPMI 1640 medium supplemented with 10% FBS and 100 μg/ml endothelial cell growth supplement (BD Biosciences).

Fibroblasts are grown under the same conditions used to maintain fetal fibroblasts as described above.

### Example 4

### Evaluation of Response to Transqenic Xenograft

Porcine kidneys are obtained from transgenic pigs. Rhesus macaques receive donor porcine kidneys following bilateral nephrectomy. Anti-pig IgG antibody titers are determined by flow cytometry analysis prior to transplant; animals with low anti-pig IgG titers are selected. T cells are depleted by treating the recipient monkeys with one dose anti-CD4/anti-CD8. Costimulation blockade is performed using either Fc-intact anti-CD154 (5C8, n=3) or belatacept (n=2). Monkeys are treated with MMF and steroids as well. An immunosuppressive compound of interest is administered to experimental monkeys with transgenic porcine kidneys and control monkeys with wildtype porcine kidneys. Clinical indicia of a side effect are monitored.

### Example 5

### Transqenic Piqs Lackinq a Cellular Transport Genes

A cellular transport gene locus is targeted for genome editing. Porcine fetal fibroblasts are transfected with sgRNA targeted to a cellular transport gene as described above herein according to the manufacturer's instructions.

SCNT is performed using in vitro matured oocytes (De Soto Biosciences Inc, St. Seymour Tenn. and Minitube of America (Mount Horeb, Wis.) as described in Estrada et al (2007) Cloning Stem Cells 9:229-236, herein incorporated by reference. Cumulus cells are removed from the oocytes by pipetting in 0.1% hyaluronidase. Oocytes with normal morphology and a visible polar body are selected and incubated in manipulation media (calcium-free NCSU-23 with 5% fetal bovine serum (FBS) containing 5 μg/ml bisbenzimide and 7.5 μg/ml cytochalasin B for 15 minutes. Following this incubation period, oocytes are enucleated by removing the first polar body and metaphase II plate. Single cells of site targeted cellular transport genee -/- cells are injected into each enucleated oocyte. Electrical fusion is induced with a BTX electroporator (Harvard Apparatus, Holliston Mass.). Enucleated oocytes injected with a cell (couples) are exposed to two DC pulses of 140 V for 50 μs in 280 mM mannitol, 0.001 mM CaCl₂ and 0.05 mM MgCl₂. After activation the oocytes are placed in NCSU-23 medium with a 0.4% bovine serum albumin (BSA) and incubated at 38.5° C., 5% CO₂ in a humidified atmosphere for less than one hour. Within an hour after activation, oocytes are transferred into a recipient pig.

Recipient pigs are synchronized occidental pigs on their first day of estrus. Pregnancies are verified by ultrasound approximately day 25 or day 26 after embryo transfer. Approximately thirty-two days after embryo transfer, fetuses are harvested. Fibroblasts from the fetuses are analyzed by staining with an antibody specific to the cellular transplant gene of interest or a negative isotype control. Fetal fibroblasts are used in a second round of SCNT and pregnancies are allowed to culminate in production of viable litters of genetically modified pigs.

All animals used in this study are approved by an institutional biosafety committee (IBC) and institutional animal care and use committee (IACUC).

### Example 6

### Genotype Analysis of Cellular Transport -/- Products

Genomic DNA is isolated from pig cells using the Qiamp DNA minikit (Qiagen). RNA samples are isolated using the RNeasy Plus mini kit (Qiagen) following the manufacturer's protocol. RNA quality and quantity are affirmed by Agilent bioanalyzer analysis. RNA samples are reverse transcribed using a OneStep RT-PCR kit. PCR products are purified and ligated into the pCR4-TOPO TA (Invitrogen). Transformed bacteria are plated on Luria-Bertani agar containing 50 μg/ml kanamycin for clone selection. Plasmids are isolated using the QlAprep Spin Miniprep kit (Qiagen). Nucleotide sequences are performed by the Sanger method using custom sequencing service and appropriate primers.

### Example 7

### Ex vivo Perfusion of Human Platelets through Transgenic Liver

A transgenic cellular transport gene deficient pig is anesthetized and intubated. A midline abdominal incision is made. The liver is removed and placed in a perfusion device under normothermic conditions. A continuous perfusion circuit contains a heated buffer reservoir, three pumps (1, continuous venous retrun; 2 pulsatile arterial supply; 3 continuous portal vein supply), an oxygenator (O₂), two bubble traps (BT) and flow (F) and pressure (P) monitors. The system is computer controlled to maintain perfusion with specific parameters. Humidity, temperature and air flow are maintained in the perfusion device. The perfusion device maintains constant pressure by varying the flow rate. Centrifugal flow through the portal vein and pulsatile flow through the hepatic artery are used. Both flow rates are set at porcine physiological pressure. The base perfusion solution is an oxygenated Ringers solution with physiologic nutrition and insulin.

Human platelets are obtained from healthy volunteer subjects or purchased commercially less than six days from isolation and are stored at 20-24° C. Approximately 1×10¹¹ human platelets are washed in sterile phosphate buffered saline (PBS) containing the anti-coagulant citrate dextrose. Platelets may be labeled with CFSE according to the manufacturer's protocol.

Pig livers are perfused two hours prior to the addition of platelets. Platelet samples are obtained prior to addition to the perfusion system and after the addition of the platelets at pre-determined time points. Platelet levels in the pre-perfusion and post-perfusion samples are evaluated. Pre and post-perfusion evaluation of the pig liver are performed. Wild-type pig livers are obtained, and the livers are perfused under similar conditions.

### Example 8

### Evaluation of Response to a Transqenic Xenoqraft

Porcine livers are obtained from a transgenic pig. The livers are surgically transplanted into a recently deceased human cadaver using the piggyback method. An immunosuppressive compound is distributed throughout the cadaver. After the surgery, biological samples are obtained from the human cadaver. Clinical indicia of side effects to the immunosuppressive agent are monitored.

### Example 9

### Evaluation of Response to a Transqenic Xenoqraft

Porcine kidneys are obtained from a transgenic pig. A highly sensitized human subject is administered immunosuppressive compounds to manage the rejection response. The porcine kidneys are surgically transplanted into the subject. After the surgery, biological samples are obtained. Clinical indicia of a side effect of the immunosuppressive compound are monitored.

### Example 10

### Confocal Microscopy Analysis

Piglets (transgenics, wild type or other piglets of interest) are euthanized. Liver, heart and kidney tissue are obtained from the pig. Frozen sections of each tissue are prepared. Mounted tissues are blocked in Odyssey blocking buffer (Li-Cor Biosciences, Lincoln Neb.) in HBSS for one hour. The slides are fixed in 4% paraformaldehyde for 10 minutes. Tissues are stained with labeled antibodies to visualize the presence of the cellular transport gene product. Tissues are washed three times with HBSS. Secondary antibody is incubated with the tissue for approximately an hour. Tissues are washed three times with 0.1% HBSS Tween. To stain the nucleus, DAPI stain (Invitrogen, Grand Island N.Y.) is added to all the slides for 1 minute followed by two 0.1% HBSS Tween washes. Tissues are mounted in ProLong Gold (Invitrogen, Grand Island N.Y.). Confocal microscopy is performed using an Olympus FV1000.

### Example 11

### Porcine Liver Procurement

Pigs are premedicated, intubated and anesthetized with propofol and placed in the supine position. A midline incision to the abdomen is made. Ligamentous attachments to the liver are taken down. The portal vein and hepatic artery are cannulated and flushed with 2 liters of cold histidine-tryptophan-ketoglutarate solution (Essential Pharmaceuticals, LLC). Livers are removed from pigs and stored in histidine-tryptophan-ketoglutarate solution on ice at 4° C. until being placed in a liver perfusion circuit. Cold-ischemia time varies between 45 minutes to 3 hours. In certain experiments porcine livers may be obtained from abbatoirs. Porcine livers from abbatoirs are flushed with histidine-tryptophan-ketoglutarate solution containing heparin (2000 U/L) within two minutes of exsanguinations.

### Example 12

### Kidney Xenograft in NHP

Recipient non-human primates (NHP) are treated with one dose of anti-CD4/anti-CD8, anti-CD154/anti-CD28 dAbs, MMF and steroids. Rhesus macaques (Macaca mulatta) are used as the NHP. In some experiments, macaques may be 3-5 years old and less than 6 kg. Transgenic porcine kidneys (or wildtype control kidneys) are transplanted into the NHP recipients. Samples (blood, urine and kidney biopsy samples) are collected at defined time points for analysis. Renal function, serum creatinine, the presence and quantity of xenoantibodies (flow cytometry and multi-parameter flow cytometry), cytokine secretion, transcript profiles from peripheral blood, urine and graft biopsies, xenograft histology and development of anti-pig antibody (flow-based xenocrossmatch assay) are followed. The CMAH deletion is not helpful for study in NHP. For NHP studies, pigs with a wild-type CMAH gene are used. Ultrasound guided needle biopsies are performed at 2, 5 and 10 weeks post transplant.

### Example 13

### Veterinary Care of NHP

NHP are housed in individual cages and provided with clean, adequately sized living quarters; fed twice daily; and are checked at least twice daily by animal care technicians and once daily by clinical veterinary staff. Physical examinations are performed each time an animal is anesthetized for blood collection or other procedures.

### Example 14

### NHP Phlebotomy and Tissue Sampling

Phlebotomy and tissue sampling (for example: blood collections, lymph node biopsies and bone marrow aspirates) of NHP's are performed either under ketamine (10 mg/kg) or Telazol (4 mg/kg) anesthesia on fasting animals. Buprenephrine (0.01 mg/kg every 6 hrs) is administered as post-operative analgesia for animals undergoing renal transplant and as needed as determined by the attending veterinarian. Animals are monitored for “irreversible critical illness” such as but not limited to loss of 25% of body weight from baseline; complete anorexia for 4 days; major organ failure or medical conditions unresponsive to treatment such as respiratory distress, icterus, uremia, intractable diarrhea, self-mutilation or persistent vomiting, and surgical complications unresponsive to immediate intervention: bleeding, vascular graft/circulation failure, infection and wound dehiscence.

### Example 15

### Porcine Embryo Transfer Surgery, Phlebotomy and Harvesting Procedures

Embryo transfer surgery: Before surgery, the sow is anesthetized with TKX (Telazol (500 mg)+Ketamine (250 mg) and Xylazine (250 mg); 1 cc per 50 lbs, IM) for intubation plus isoflurane by inhalation through ET tube using a precision vaporizer and waste gas scavenging. During the recovery period, animals are monitored at least once every 15 minutes and vital signs (temperature, heart rate, respiration rate and capillary refill time) are assessed and recorded. Trained animal care technicians or veterinarians monitor the animals until they can maintain themselves in voluntary sternal recumbrance. Animals are returned to regular housing areas upon approval by the attending veterinarian. Post-operative analgesics include buprenorphine 0.01-0.05 mg/kg IM every 8-12 hours or carprofen 2-4 mg/kg SC daily. Approximately 26 days after embryo transfer, ultrasound is performed to confirm establishment of pregnancy while the sow is distracted by food. About 10 days later a second ultrasound is performed. Birth occurs through natural parturition unless clinical difficulty arises. Caesarian section is performed recommended by the veterinary staff. Standard caesarian section protocols are used with the general anesthesia protocol utilized in the embryo transfer surgery. Experimental piglets are cleaned and the umbilical cord is disinfected. Every piglet receives colostrum during the first hours after birth. Piglets are watched 24/7 until they are at least 7 days old. Farrowing crates are used to protect the piglets from their mother while maintaining the piglets ability to nurse.

All phlebotomy is performed under either ketamine (10 mg/k) or Telazol (4 mg/kg) anesthesia on fasting animals. Organ harvesting, a terminal surgical procedure, uses the anesthesia protocol (Telazol (500 mg)+ketamine (250 mg)+xylazine (250 mg); 1 cc per 50 lbs; IM) +/− pentothal (10-20 mg/kg) IV if needed for intubation and isoflurane by inhalation through ET tube using a precision vaporizer, to effect with waste gas scavenging. Swine are perfused with saline followed by removal of the heart and other tissue/organs. Alternatively swine are anesthetized with inhaled anesthetic and treated with a barbituric acid derivative (100-150 mg/kg) and a bilateral pneumothorax is performed.

### Example 16

### Liver Xenograft in NHP

Rhesus macaques are treated with either an anti-CD28 dAb based immunosuppressive regimen (T cell depletion using anti-CD4/anti-CD8, single dose), anti-CD28 dAb, MMF and steroids; an anti-CD154 dAb based immunosuppressive regimen (T cell depletion using anti-CD54/anti-CD8, single dose, anti-CD154 dAb, MMF and steroids) or both regimens; one or more additional immunosuppressive agents may be evaluated. Livers from transgenic pigs are transplanted into rhesus macaques treated with the indicated immunosuppressive regimen. Liver biopsies are performed at 1 hour, 1 week, 4 weeks and at times of liver graft dysfunction. Laboratory assessments include CBC with platelet count and coagulation studies; liver function tests are obtained at 6 hours after transplant, every other day for 1 week and then twice weekly as well as at any time of clinical deterioration. Clinical indicia of side effects are monitored. Rejection is characterized using standard diagnostic criteria and immunohistochemistry. NHPs express CMAH, thus the CMAH deletion is not helpful in NHP studies. Some NHP studies are performed with wildtype CMAH pig tissues.

The invention is not limited to the embodiments set forth herein for illustration but includes everything that is within the scope of the claims. Having described the invention with reference to the exemplary embodiments, it is to be understood that it is not intended that any limitations or elements describing the exemplary embodiments set forth herein are to be incorporated into the meanings of the patent claims unless such limitations or elements are explicitly listed in the claims. Likewise, it is to be understood that it is not necessary to meet any or all of the identified advantages or objects of the invention disclosed herein in order to fall within the scope of any claims, since the invention is defined by the claims, and since inherent and/or unforeseen advantages of the present invention may exist even though they may not be explicitly discussed herein.

Furthermore all references cited herein are hereby incorporated by reference in their entirety and for all purposes as if fully set forth herein.

That which is claimed:

1. A transgenic pig comprising a disrupted cellular transport gene in the nuclear genome of at least one cell of said pig, wherein expression of said cellular transport gene is decreased as compared to a wild-type pig.

2. A porcine organ, tissue or cell isolated from said transgenic pig of claim 1.

3. The porcine organ, tissue or cell of claim 2, wherein said porcine organ, tissue or cell is selected from the group consisting of skin, heart, liver, kidneys, lung, pancreas, thyroid, small bowel and components thereof.

4. A transgenic pig of claim 1 wherein said cellular transport gene is selected from the group comprising NCC, NHE1, NHE3, megalin, cubulin and FK506 binding protein 12 (FK506BP12).

5. The transgenic pig of claim 1 wherein when tissue from said pig is transplanted into a human and said human receives an immunosuppressive medication, a side effect of said immunosuppressive medication is improved as compared to when tissue from a wild-type pig is transplanted into a human.

6. The transgenic pig of claim 1 wherein when tissue from said pig is transplanted into a human, the side effect is selected from the group comprising high blood pressure, cardiomyopathy, renal tube dysfunction, hyperkalemia, hypercalciuria and acidosis.

7. The transgenic pig of claim 1 wherein when a heart from said transgenic pig is transplanted into a human recipient of FK506, said heart exhibits less FK506-induced cardiomyopathy than a heart from a wild-type pig.

8. The transgenic pig of claim 1 wherein when a kidney from said transgenic pig is transplanted into a human recipient of an immunosuppressive medication, said patient exhibits decreased kidney related side effects.

9. The transgenic pig of claim 5 wherein said immunosuppressive medication is selected from the group comprising calcineurin inhibitors, FK506, and cyclosporine.

10. The transgenic pig of claim 1 wherein when a kidney from said pig is transplanted into a human and said human receives gentamycin, gentamycin related nephrotoxicity is improved as compared to when a kidney from a wild-type pig is transplanted into a human.

11. A transgenic pig of claim 1 further comprising a disrupted α(1,3)-galactosyltransferase gene and a disrupted CMAH gene in the nuclear genome of at least one cell of said pig wherein expression of α(1,3)-galactosyltransferase and CMAH in said pig is decreased as compared to a wild-type pig.

12. A transgenic pig of claim 1 further comprising a disrupted ASGR1 gene in the nuclear genome of at least one cell of said pig wherein expression of ASGR1 in said pig is decreased as compared to a wild-type pig.

13. A transgenic pig of claim 1 further comprising a disrupted β4GalNT2 gene in the nuclear genome of at least one cell of said pig wherein expression of β4GalNT2 in said pig is decreased as compared to a wild-type pig.
